SEQUENCE LISTING (2 sequences)

SEQ ID NO: 1 - DNA, 20 bp, Artificial Sequence
  Synthetic forward primer
ctggccatgaactacctgga

SEQ ID NO: 2 - DNA, 20 bp, Artificial Sequence
  Synthetic reverse primer
gtcacacttgatcactctgg

(19) United States
(12) Patent Application Publication
(10) Pub. No.: US 2008/0166425 A1
(43) Pub. Date: Jul. 10, 2008

(54) METHOD FOR INHIBITING CANCER USING ARSENIC TRIOXIDE

(75) Inventor: Yok-Lam Kwong, Hong Kong (HK)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: The University of Hong Kong

(21) Appl. No.: 11/867,834

(22) Filed: Oct. 5, 2007

Related U.S. Application Data
(63) Continuation-in-part of application No. 10/669,869, filed on Sep. 23, 2003.
Application No. 10/669,869 is a continuation-in-part of application No. 11/549,347, filed on Oct. 13, 2006.
(60) Provisional application No. 60/417,200, filed on Oct. 9, 2002.
(60) Provisional application No. 60/483,014, filed on Jun. 25, 2003.

(51) Int. Cl. A61K 33/36; C12N 5/02; A61P 35/04

(52) U.S. Cl. 424/623; 435/375

(57) ABSTRACT

The invention provides a method for treating cancers that are dependent on cyclin D1 for proliferation, survival, metastasis and differentiation, involving administering a composition containing an effective amount of arsenic trioxide to an affected patient. The arsenic trioxide can be administered orally, for example, as a solution, suspension, syrup, emulsion, tablet, or capsule. The composition can also contain one or more pharmaceutically acceptable carriers and/or excipients.

### CROSS-REFERENCE TO RELATED APPLICATIONS

This application is a continuation in part of U.S. Ser. No. 10/669,869 filed Sep. 23, 2003, which claims priority to U.S. Ser. No. 60/417,200 filed Oct. 9, 2002 and U.S. Ser. No. 60/483,014 filed Jun. 25, 2003, and is also a continuation-in-part of Ser. No. 11/549,347 filed Oct. 13, 2006 and all of which are incorporated by reference in their entirety.

### FIELD OF INVENTION

This invention relates to methods of inhibiting cancer by affecting expression, translation, and biological activity of cancers overexpressing or dependent on cyclin D1 using arsenic trioxide.

### BACKGROUND OF THE INVENTION

Mantle cell lymphoma (MCL) is a well-defined subtype of B cell lymphoma in the World Health Organization classification, and accounts for approximately 3-10% of all non-Hodgkin lymphomas. The chromosomal aberration t(11;14)(q13;q32) can be found in practically all cases of MCL. The translocation results in juxtaposition of the immunoglobulin heavy chain joining region on chromosome 14 to the cyclin D1 gene on chromosome 11. The molecular consequence of the translocation is to place cyclin D1 under the control of the immunoglobulin heavy chain gene enhancer, leading to over-expression of the cyclin D1 protein.

Although MCL accounts for approximately 3-8% of B-cell lymphomas, it is difficult to manage. Initial treatment with rituximab plus combination chemotherapy or purine analogues results in complete remission (CR) rates varying from 34-87%. However, relapses occur in most patients with prolonged follow up. Treatment options for relapsed patients are limited. Several approaches have been adopted, including the use of the proteasome inhibitor bortezomib, thalidomide and the mammalian target of rapamycin (mTOR) inhibitor temsirolimus. The overall response (OR) rates of these agents varied from 38-81%, but the CR rate was only 3-31%. Therefore, there is an urgent need to define effective treatment strategies for MCL.

It is an object of this invention to provide agents and methods for treating cancers such as MCL and other cancers over-expressing cyclin D1.

It is another object of this invention to provide methods, strategies, doses, and dosing schedules for the administration of As₂O₃ in the clinical inhibition of cancers over-expressing cyclin D1.

### SUMMARY OF THE INVENTION

It has been discovered that As₂O₃ suppresses cyclin D1 and initiates down-regulation of cyclin D1 by activating GSK-30, which phosphorylates cyclin D1. Activation of IKKb leads to phosphorylation of cyclin D1, which is ubiquitinated. Ubiquitinated cyclin D1 is degraded in the proteasome. This is the basis for the discovery that MCL and other cancers over-expressing cyclin D1 can be treated with As₂O₃, preferably oral As₂O₃.

### BRIEF DESCRIPTION OF DRAWINGS

FIG. 1A is a line graph showing As₂O₃ (concentration in micromolar) percent induced apoptosis in MCL cells, based on a MTT test of Jeko-1 and Granta-519 cells treated for 72 hours with As₂O₃. There was a dose and time dependent suppression of cellular proliferation. Viability significantly decreased at or above 1 μM As₂O₃ as compared with baseline (one-way ANOVA with Dunnett's post-tests, p<0.05) (triplicate experiments)

FIG. 1B is a scatter plot of popidium iodide versus annexin expression in cells treated with As₂O₃. There was a significant increase in apoptotic cells after As₂O₃ treatment. (#: apoptotic cells that were annexin V positive and popidium iodide negative).

FIG. 2 shows down-regulation of cyclin D1 by As₂O₃ treatment. FIG. 2A: As₂O₃ (4 μM) induced a time dependent down-regulation of cyclin D1 in Jeko-1 and Granta-519 cells. Triplicate experiments and a representative Western blot demonstrate significant decrease in cyclin D1 level after 2 hours (one-way ANOVA with Dunnett's post-tests, p<0.05). FIG. 2B: As₂O₃ (treatment for 8 hours) induced a dose dependent down-regulation of cyclin D1 in Jeko-1 and Granta-519 cells. Triplicate experiments demonstrate significant decrease in cyclin D1 level at or above 2 μM (one-way ANOVA with Dunnett's post-tests, p<0.05).

FIG. 3 shows dephosphorylation of retinoblastoma (RB) by As₂O₃ treatment in MCL lines. As₂O₃ treatment resulted in dephosphorylation of RB (significant decrease of phosphor-Rb Ser-795 at or more that 8 hours of As₂O₃ treatment, triplicate experiments, one-way ANOVA with Dunnett's post-tests, p<0.05).

FIG. 4 shows As₂O₃ treatment induced phosphorylation of cyclin D1 and GSK-3. A. Cell lysates immunoblotted with anti-phospho-cyclin D1 (Thr-286) As₂O₃ treatment led to significantly increased phosphor-cyclin D1 (triplicate experiments, one-way ANOVA with Dunnett's post-tests, p<0.05). B. Cell lysates immunoblotted with anti-phospho-cyclin GSK-3β (Try-216). As₂O₃ treatment led to significantly increased phosphor-GSK-3β (triplicate experiments, one-way ANOVA with Dunnett's post-tests, p<0.05). C. Pre-incubation with 6-bromoindirubin-3′-oxime (BIO; 10 μM) before As₂O₃ treatment (4 μM, 8 hour, 37° C.) prevented cyclin D1 down-regulation, showing that GSK-3b was involved. Result a significant reduction of cyclin D1 as compared with control (triplicate experiments, one-way ANOVA with Dunnett's post-tests, p<0.05).

FIG. 5 shows that IKK was involved in As₂O₃-induced down-regulation of cyclin D1. A. As₂O₃ treatment (4 μM for 2 hours) led to a significant increase in phosphor-IKKα/β (Ser-176/180) (triplicate experiments, one-way ANOVA with Dunnett's post-tests, p<0.05). B. Pre-incubation with the IKK inhibitor BMS (10 mM, 30 minutes) successfully prevented As₂O₃-induced cyclin D1 down-regulation (triplicate experiments, one-way ANOVA with Dunnett's post-tests, p<0.05).

FIG. 6 shows As₂O₃ induced ubiquitination of cyclin D1 in MCL. A. Cell lysates were immunoprecipitation with anti ubiquitin (Ub) or anti-cyclin D1 antibody. The immunoprecipitates and the crude lysates were immunoblotted with anti-cyclin D1 and anti-ubiquitin antisera (FIG. 6B). As₂O₃ induced a significant increase in binding between cyclin D1 and ubiquitin (increase in ubiquitination from 30 minutes to 2 hours after As₂O₃ treatment as compared to the baseline, triplicate experiments, one-way ANOVA with Dunnett's post-tests, p<0.05).

FIG. 7 shows As₂O₃-induced cyclin D1 degradation involved the proteasome but not the lysosome in MCL. A. Pre-incubation with the proteasome inhibitors MG132 (MG, 30 μM), bortezomib (bort, 10 μg/ml) and lactacystin (lact, 10 μM) successfully prevented As₂O₃ induced cyclin D1 degradation. B. Pre-incubation with the lysosomal inhibitor ammonium chloride (NH₄Cl, 2.5 mM) was ineffective in preventing As₂O₃-induced cyclin D1 degradation.

FIG. 8 is a schematic diagram showing the proposed mechanism of degradation of cyclin D1 mediated by As₂O₃.

### DETAILED DESCRIPTION OF THE PREFERRED EMBODIMENTS

### I. Arsenic Trioxide Formulations

Arsenic Trioxide

Arsenic trioxide is available from a number of different suppliers. Arsenic trioxide is an amphoteric oxide which is known for its acidic properties. It dissolves readily in alkaline solutions to give arsenites. It is much less soluble in acids, but will dissolve in hydrochloric acid to give arsenic trichloride or related species. It reacts with oxidizing agents such as ozone, hydrogen peroxide and nitric acid to give arsenic pentoxide, As₂O₅. It is also readily reduced to arsenic, and arsine (AsH₃) may also be formed.

Arsenic trioxide has many uses including as: a starting material for arsenic-based pesticides; a starting material for arsenic-based pharmaceuticals, such as a neosalvarsan, a synthetic organoarsenic antibiotic; a decolorizing agent for glasses and enamels, a wood preservative, and a cytostatic in the treatment of refractory promyelocytic (M3) subtype of acute myeloid leukemia.

An oral arsenic trioxide (As₂O₃) is highly efficacious for relapsed acute promyelocytic leukemia. Oral As₂O₃ causes a smaller prolongation of QT intervals, and therefore is a much safer drug for treating leukemia.

Formulations

The following delivery systems, which employ a number of routinely used pharmaceutical carriers, are only representative of the many embodiments envisioned for administering the instant compositions.

Parenteral Formulations

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Enteral Formulations

Oral delivery systems include solid dosage forms such as tablets (e.g, compressed tablets, sugar-coated tablets, film-coated tablets, and enteric coated tablets), capsules (e.g., hard or soft gelatin or non-gelatin capsules), blisters, and cachets. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). The solid dosage forms can be coated using coatings and techniques well known in the art.

Oral liquid dosage forms include solutions, syrups, suspensions, emulsions, elixirs (e.g., hydroalcoholic solutions), and powders for reconstitutable delivery systems. The formulations can contain one or more carriers or excipients, such as suspending agents (e.g., gums, zanthans, cellulosics and sugars), humectants (e.g., sorbitol), solubilizers (e.g., ethanol, water, PEG, glycerin, and propylene glycol), surfactants (e.g., sodium lauryl sulfate, Spans, Tweens, and cetyl pyridine), emulsifiers, preservatives and antioxidants (e.g., parabens, vitamins E and C, and ascorbic acid), anti-caking agents, coating agents, chelating agents (e.g., EDTA), flavorants, colorants, and combinations thereof. The compositions can be formulated as a food or beverage (e.g., a shake) containing buffer salts, flavoring agents, coloring agents, sweetening agents, and combinations thereof.

Topical Formulations

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

### II. Methods of Treatment

Cyclin D1 is a D-type cyclin critically involved in the control of the cell cycle. It assembles with its catalytic partners cyclin-dependent kinase 4 (CDK4) and CDK6 to form an active holoenzyme complex, which controls G1 progression and G1/S transition. The active holoenzyme complex phosphorylates the retinoblastoma protein RB. Phosphorylated RB releases the E2F family of transcription factors from inhibition, enabling E2Fs to coordinately regulate genes necessary for DNA replication and hence progression into S phase. Over-expression of cyclin D1 is demonstrable in many cancers, including cancers of the digestive tract, cancers of the female genital tract, and malignant lymphomas.

Owing to its important influence on the cell cycle, cyclin D1 expression is carefully regulated. Cyclin D1 gene mRNA and transcription appears to be constant through the cell cycle. However, a decline in cyclin D1 level occurs during S phase, which has been attributed to its increased proteasomal degradation. Cyclin D1 phosphorylation at a threonine residue 286 (Thr-286) positively regulates its proteasomal degradation. Thr-286 phosphorylation is mediated by glycogen synthase kinase-3β (GSK-3β). In addition to targeting cyclin D1 to proteosomes, GSK-3β-induced Thr-286 phosphorylation also promotes cyclin D1 nuclear export, by increasing the binding of cyclin D1 to a nuclear exportin CRM1. IkappaB kinase (IKK) alpha, IKKα, associates with and phosphorylates cyclin D1 also at Thr-286, thereby participating in the subcellular localization and turnover of cyclin D1.

As₂O₃ induced apoptosis in MCL lines at 2-4 μM, which is within the plasma levels achieved after As₂O₃ therapy. As₂O₃ induces a dose and time dependent suppression of cyclin D1. The suppression of cyclin D1 restores RB to a hypophosphorylated state, in parallel with a change in cell cycle. These biologic changes are consistent with the apoptosis observed upon As₂O₃ treatment.

The down-regulation of cyclin D1 mediated by As₂O₃ occurs at a post-transcriptional level since cyclin D1 is under the transcriptional control of the immunoglobulin heavy chain gene enhancer in MCL, which is unlikely to be affected by As₂O₃. Furthermore, in physiologic conditions, the control of cyclin D1 during the cell cycle is also mediated in part via alteration in the stability of cyclin D1. This process is controlled by phosphorylation of cyclin D1 at Thr-286, a process mediated by GSK-3β. GSK-3β is itself tightly regulated. Mitogens inactivate GSK-3β by a pathway involving Ras, phosphatidylinositol 3 kinase (PI3K), and protein kinaseB/Akt. Ras activates PI3K, which in turn activates Akt. Akt inactivates GSK-3β by phosphorylating it at serine residue 9. This removes the inhibition of GSK-3β on cyclin D1, allowing cyclin D1 to accumulate and thus activate cell cycling. GSK-3β can also be activated by phosphorylation at a tyrosine residue 216 (Try-216) in the kinase domain. As₂O₃-mediates an increase of GSK-3β Try-216 phosphorylation. The end result of As₂O₃-mediated increase in GSK-3β Try-216 phosphorylation is the increase in cyclin D1 Thr-286 phosphorylation, a key step in its degradation.

The IKK complex is the major regulatory component in the NK-κB pathway. It comprises the catalytic subunits IKKα and IKKβ, and a regulatory subunit IKKγ/NEMO. IKKα has been shown to phosphorylate cyclin D1 at Thr-286, the same site targeted by GSK-3b. IKKα needs to be activated by phosphorylation at a serine residue 176 (Ser-176) before participating in the regulation of NF-κB by phosphorylating IκB. IKKα Ser-176 phosphorylation is mediated by NK-κB inducing kinase (NIK). As₂O₃-induces an increase in IKK phosphorylation. As₂O₃-mediates an increase in physical interaction between IKK and cyclin D1, as shown in immunoprecipitation experiments. An IKK specific inhibitor BMS-345541 alleviated As₂O₃-induced cyclin D1 down-regulation. These results indicate that IKK is also an effector of As₂O₃ treatment.

As₂O₃-mediated cyclin D1 Thr-286 phosphorylation increases its ubiquitination. The time course of ubiquitination is commensurate with the timing of the biologic functions of As₂O₃ on the MCL lines. After As₂O₃ treatment, increased ubiquitination is first detected at 30 minutes and continues to increase. At two hours, significant down-regulation of cyclin D1 is first observed, which is associated with a parallel hypophosphorylation of RB. Significant activation of caspase 3 is observed at four hours. These sequence of events are consistent with cyclin D1 down-regulation initiated by Thr-286 phosphorylation.

Cyclin D1 is a cytosolic and nuclear protein. Therefore, polyubiquitination is involved, which targets the protein to degrade in proteasomes. Inhibition of proteasomes successfully prevented As₂O₃-induced down-regulation of cyclin D1. Inhibition of lysosomes, the site of degradation of monoubiquitinated proteins, does not interfere with As₂O₃-induced down-regulation of cyclin D1. These results confirm that As₂O₃ down-regulated cyclin D1 by promoting its proteasomal degradation.

Arsenic trioxide can be used for the treatment of cancers that are dependent on cyclin D1 for proliferation, survival, metastasis and differentiation.

Patients with cancers that overexpress cyclin D can be treated with As₂O₃. Mantle cell lymphoma is a cancer characterized by overexpression of cyclin D, as are cancers of the digestive tract, cancers of the female genital tract, and malignant lymphomas.

The dose of oral As₂O₃ is typically adjusted according to age and kidney function. In one embodiment, the dose range of As₂O₃ varies from 1 to 10 mg, typically about 5 to 10 mg.

The present invention will be further understood by reference to the following non-limiting examples.

### EXAMPLES

### Example 1

### In Vitro Studies Show As₂O₃ is Effective in Treatment of MCL by Targeting Cyclin D1

### Materials and Methods

Cell lines. The MCL lines Jeko-1 and Granta-519 were obtained from German Collection of Microorganisms and Cell Cultures (ACC 553 and ACC 342, Braunschweig, Germany). Jeko-1 cells were cultured in RPMI 1640 with 20% fetal bovine serum (FBS), and Granta-519 cells in DMEM with 10% FBS; both with 50 units/ml penicillin and 50 μg/ml streptomycin, at 5% C0₂.

Reagents and antibodies. Reagents and antibodies used included cell culture reagents (Invitrogen, Carlsbad, Calif., USA); kinase inhibitors and their inactive analogues (Calbiochem, Darmstadt, Germany); antiserum to phospho-GSK3 (tyrosine 216, Try-216) (Upstate, Lake Placid, N.Y., USA); antisera to cyclin D1, phospho-cyclin D1 (Thr-286), GSK3β, phospho-GSK3β (Tyr-216), IκB kinase (IKK)α/β, phospho-IKKα/β (serine 176/180, Ser-176/180), RB and phospho-RB (serine 795, Ser-795), caspase-3 and β-actin (Cell Signaling Technology, Beverly, Mass., USA); protein G-agarose (Upstate); ECL kit (Amersham, Piscataway, N.J., USA); cell proliferation kit I (MTT) (Roche Applied Science, Indianapolis, Ind., USA); annexin V-FITC Kit (Beckman Coulter, Fullerton, Calif., USA); and RNeasy Kit and One-Step RT-PCR Kit (Qiagen, Valencia, Calif., USA).

Cell viability assays. Cells were seeded on 96-well microplates at 2×10⁴/well in 100 ml growth medium containing different concentration of As₂O₃ as indicated at 37° C. for 72 hours. MTT labeling reagent (10 μl, 5 mg/ml) (Roche Applied Science, Indianapolis, Ind., USA) was added to each well at 37° C. for 4 hours, followed by 100 μl solubilization at 37° C. overnight. Solubilized fomarzan crystals were quantified spectophotometrically at 590 nm with a microplate ELISA reader.

Apoptosis assay. Cells were seeded at 1×10⁶/ml in different concentrations of As₂O₃ as indicated at 37° C. for 24 hours, harvested, rinsed in ice-cold phosphate buffered saline (PBS), and resuspended in 500 μl binding buffer containing annexin V-FITC and propidium iodide (PI) (Beckman Coulter, Fullerton, Calif., USA) for 20 minutes on ice. The percentages of apoptotic cells (annexin-V positive, PI negative) were determined on a flow cytometer (Epics, Beckman Coulter) with appropriate color compensation.

Cell Cycle Analysis. Cells were seeded at 1×10⁵/ml in different concentrations of As₂O₃ as indicated at 37° C. for 8 hours, harvested, washed in ice-cold PBS, resuspended in 500 μl PBS, stained with PI for 10 minutes on ice. Cell cycle was determined by flow cytometry (Epics, Beckman Coulter).

Semi-quantitative reverse transcription polymerase chain reaction (RIT-PCR) for cyclin D1. Cells were seeded at a density of 1×10⁶/ml in different concentrations of As₂O₃ at 37° C. for 8 hours, washed with PBS buffer and lysed with RTL buffer. RNA was extracted with an RNeasy Kit, followed by cDNA synthesis and a 30-cycle PCR with a One-Step RT-PCR Kit with the forward primer 5′-CTG GCCC ATG AAC TAC CTG GA-3′ and the reverse primer 5′-GTC ACA CTT GAT CAC TCT GG-3′. Cycling conditions were denaturation (1 minute at 94° C., first cycle 5 minutes), annealing (2 minutes at 50° C.) and extension (3 minutes at 72° C., last cycle 10 minutes).

Western Blotting Analysis. Cells were seeded at a density of 1×10⁶/ml overnight. Where applicable, cells were pre-treated with various inhibitors for 30 minutes, and then incubated with 4 μM As₂O₃ for different time periods as indicated. Cells were lysed in lysis buffer (50 mM Tris-HCl, 100 mM NaCl, 5 mM EDTA, 40 mM NaP₂O₇, pH 7.5, 1% Triton X-100, 4 μg/ml aprotinin, 1 mM dithiothreitol, 200 μM Na₃VO₄, 0.7 μg/ml pepstatin, 100 μM phenylmethylsulfonyl fluoride, and 2 μg/ml leupeptin). Clarified lysates were resolved on 12% SDS-phenylmethylsulfonyl fluoride and transferred to nitrocellulose membranes. The membranes were blocked with 5% non-fat milk, washed, incubated with the appropriate antibodies followed by horseradish peroxidase-conjugated secondary antisera. Immuno-reactive bands were visualized by chemiluminescence with the ECL kit, detected on X-ray films and quantified by densitometric scanning (Eagle Eye II still video system, Stratagene, La Jolla, Calif., USA).

Coimmunoprecipitation Assays. Cells were seeded at 1×10⁶/ml overnight, treated with 4 μM As₂O₃ at 37° C. for different time periods as indicated, and lysed in lysis buffer. Cell lysates were incubated with an anti-cyclin D1, anti-ubiquitin, anti-calpain 2 or anti-IKKα/β antibodies (4 μg/sample) at 4° C. for 1 hour, followed by incubation with 30 μl of protein G-agarose (50% slurry) at 4° C. for another 2 hour. Immunoprecipitates were washed four times with 400 μl lysis buffer, resuspended in 50 μl lysis buffer and 10 ml 6×sample buffer and boiled for 5 minutes. Immunoprecipitates were then analysed by Western blot analysis.

### Results

As₂O₃ induced dose and time dependent apoptosis in MCL cells.

The MTT test showed that As₂O₃ induced a dose-dependent cytotoxicity in Jeko-1 and Granta-519 cells. Flow cytometric analysis showed that As₂O₃ treatment led to induction of apoptosis. Western blot analysis showed that caspase 3 activation was involved in As₂O₃-induced apoptosis.

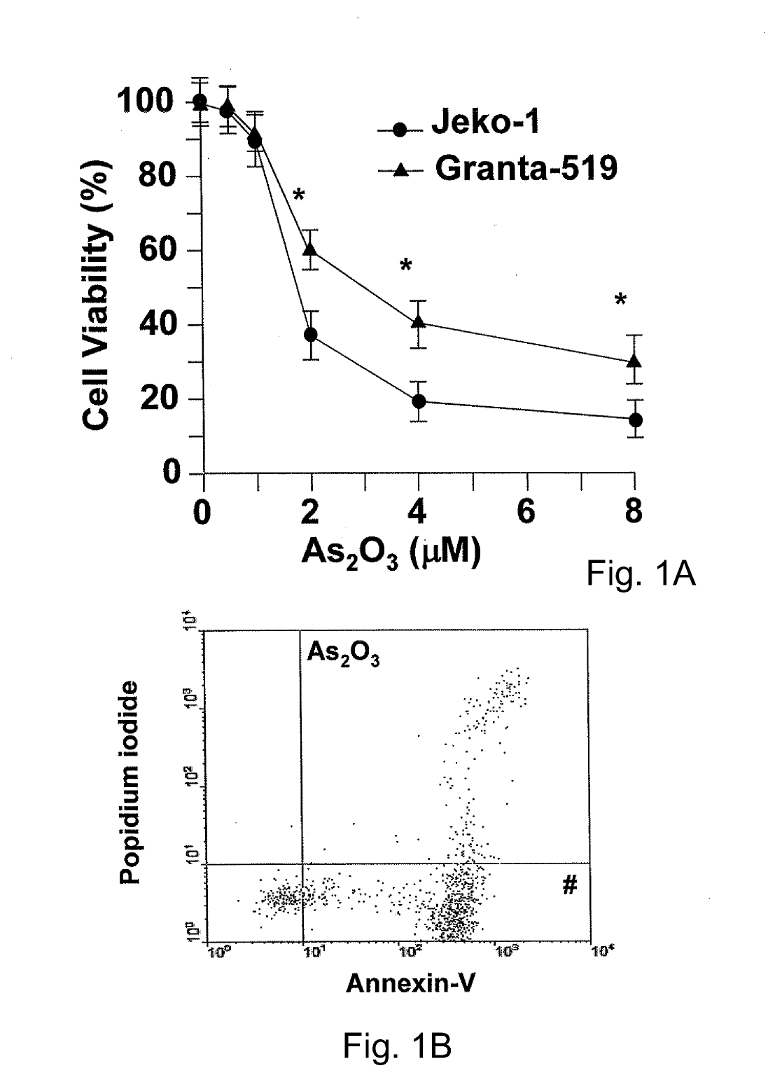

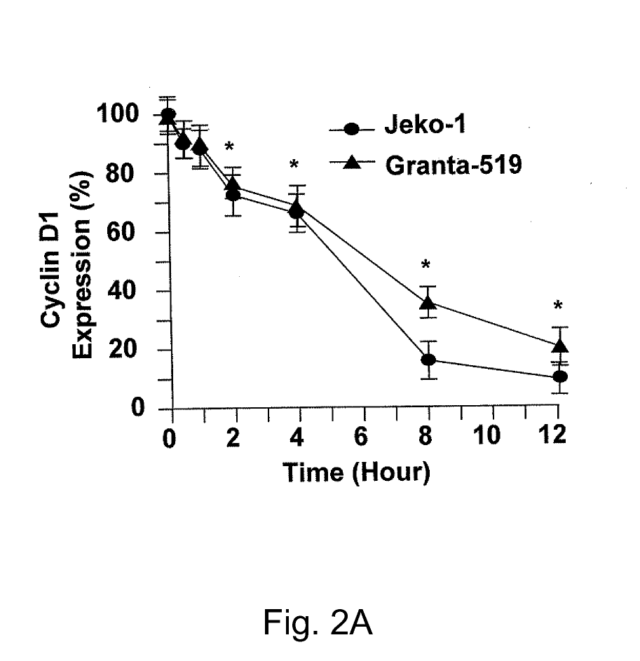

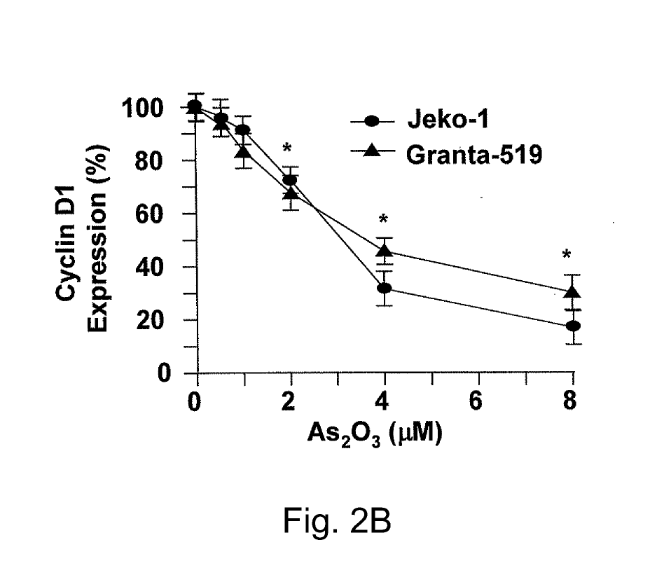

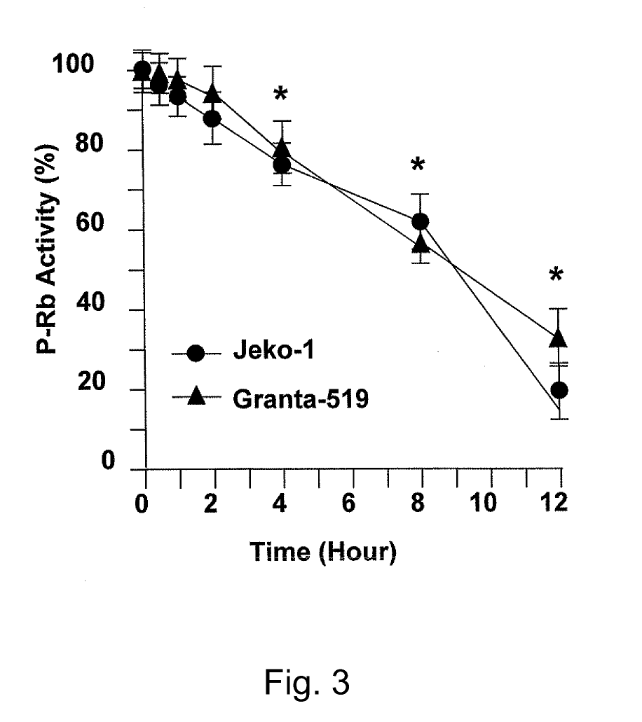

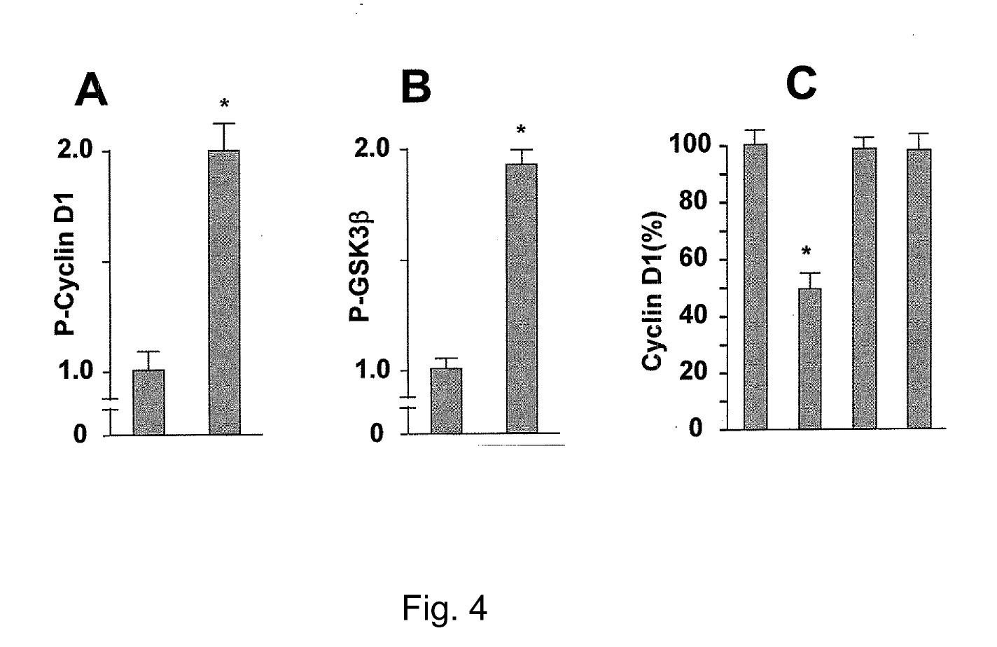

FIGS. 1A and 1B are graphs showing As₂O₃ (concentration in microM) percent induced apoptosis in MCL cells measured using a MTT test of Jeko-1 and Granta-519 cells treated for 72 hours with As₂O₃. There was a dose and time dependent suppression of cellular proliferation. Viability significantly decreased at or above 1 μM As₂O₃ as compared with baseline (one-way ANOVA with Dunnett's post-tests, p<0.05) (triplicate experiments). (Significant increase in apoptotic cells after As₂O₃ treatment. #: apoptotic cells that were annexin V positive and popidium iodide negative). Western Blotting showed activation of caspase 3 by As₂O₃ treatment, 0, 1.5 and 2.5 microM. Cleaved caspase 3 were detectable four hours after As₂O₃ treatment.

Cyclin D1 was down-regulated in MCL by As₂O₃. To determine the molecular mechanisms of As₂O₃-induced apoptosis in MCL, the expression of cyclin D1 was examined. Western blot analysis showed that As₂O₃-induced a time and dose dependent suppression of cyclin D1 in both Jeko-1 and Granta-519 cell lines. Treatment with As₂O₃ at 4 μM led to suppression of cyclin D1, first detectable at 2 hours and almost complete at 8-12 hours As₂O₃ suppression of cyclin D1 was also dose-dependent. Triplicate experiments demonstrate significant decrease in cyclin D1 level after 2 hours (one-way ANOVA with Dunnett's post-tests, p<0.05) Triplicate experiments demonstrate significant decrease in cyclin D1 level at or above 2 μM (one-way ANOVA with Dunnett's post-tests, p<0.05). Semi-quantitative polymerase chain reaction showing that cyclin D1 gene transcription was unaffected by As₂O₃ treatment.

As₂O₃ induced down-regulation of cyclin D1 disrupted its signaling. To investigate if cyclin D1 down-regulation is biologically relevant, RB phosphorylation was investigated. As₂O₃ treatment led to a time dependent decrease in RB phosphorylation, which occurred at a similar time-frame as compared with cyclin D1 down-regulation. Cell cycle analysis by flow cytometry showed that there was an increase in the proportion of apoptotic cells.

Down-regulation of cyclin D1 by As₂O₃ was post-transcriptional. RT-PCR showed that cyclin-D1 gene transcription was unaffected by As₂O₃ treatment of up to 8 μM, suggesting that the down-regulation of cyclin D1 was post-transcriptional.

As₂O₃-induced cyclin D1 down-regulation was related to GSK3β activation. Western blot analysis showed that As₂O₃ treatment resulted in significant increases in cyclin D1 phosphorylation at Thr-286, a prerequisite for cyclin D1 degradation. Cyclin D1 phosphorylation by GSK-3β requires prior activation of GSK-3β by phosphorylation at Tyr-216. As₂O₃ treatment significantly increased GSK-3β Tyr-216 phosphorylation, indicating that GSK-3β might mediate As₂O₃-induced cyclin D1 phosphorylation and hence degradation. To confirm the role of GSK-3β as a mediator of As₂O₃, Jeko-1 cells were pre-incubated with the GSK-3β inhibitor 6-bromoindirubin-3′-oxime (BIO; 10 μM) before As₂O₃ treatment. The results showed that BIO successfully prevented As₂O₃-induced down-regulation of cyclin D1. Collectively, these observations indicate that As₂O₃ down-regulated cyclin D1 post-transcriptionally, probably by increasing its degradation.

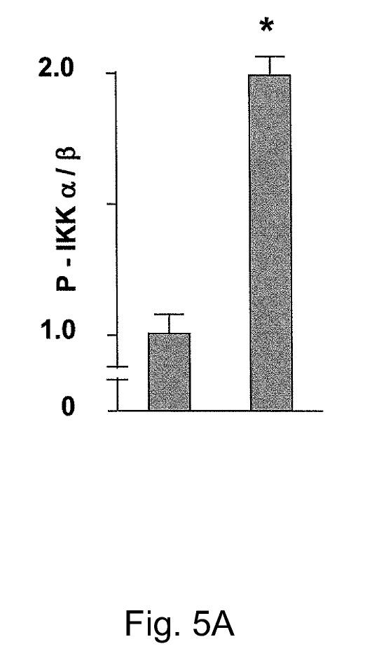

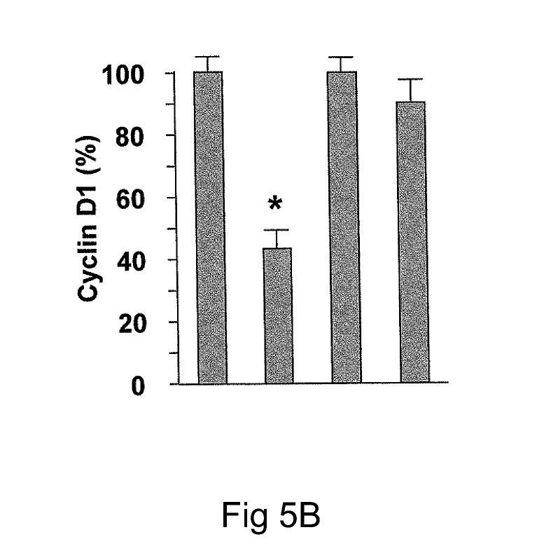

As₂O₃-induced cyclin D1 down-regulation was also dependent on IKKα/β. To determine if IKK was involved in As₂O₃-induced down-regulation of cyclin D1, IKKα/β phosphorylation at Ser-178/180 was examined. As₂O₃ significantly increased IKKα/β Ser-178/180 phosphorylation, which was required for activation of IKKα/β (FIG. 5A). Pre-treatment with the IKK(α/β inhibitor BMS-345541 (BMS; 10 μM) significantly prevented As₂O₃-induced cyclin D1 down-regulation, suggesting that IKKα/β was a molecular mediator of As₂O₃ (FIG. 5B). Immunoprecipitation with an anti-IKKα/β antibody showed that cyclin D1 bound IKKα/β. Similarly, when cyclin D1 was immunoprecipitated, IKKα/β was also confirmed to co-imnunoprecipitate. These results confirmed that As₂O₃ activated IKKα/β, which participated in the down-regulation of cyclin D1.

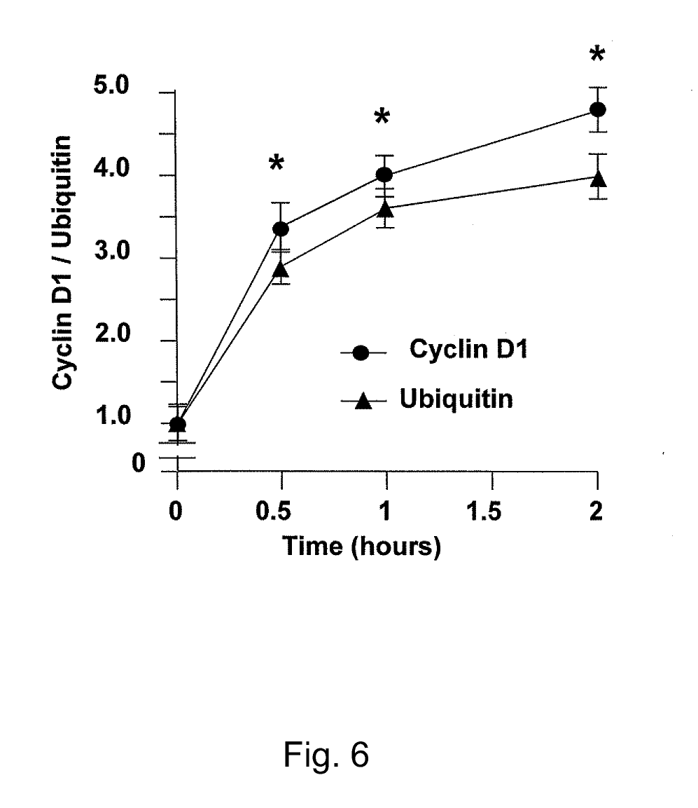

As₂O₃ promoted cyclin D1 ubiquitination. To study if As₂O₃-induced cyclin D1 down-regulation was mediated via ubiquitination, immunoprecipitation experiments were performed on lysates from Jeko-1 cells treated with As₂O₃. Immunoprecipitation with an anti-ubiquitin antibody showed a time-dependent increase in bound cyclin D1 (FIG. 6A and B) Similarly, lysates immunoprecipitated with an anti-cyclin D1 antibody also showed a time dependent increase in bound ubiquitin. These results showed that As₂O₃ promoted cyclin D1 ubiquitination, confirming that As₂O₃-induced GSK-3β and IKKα/β activation was biologically relevant.

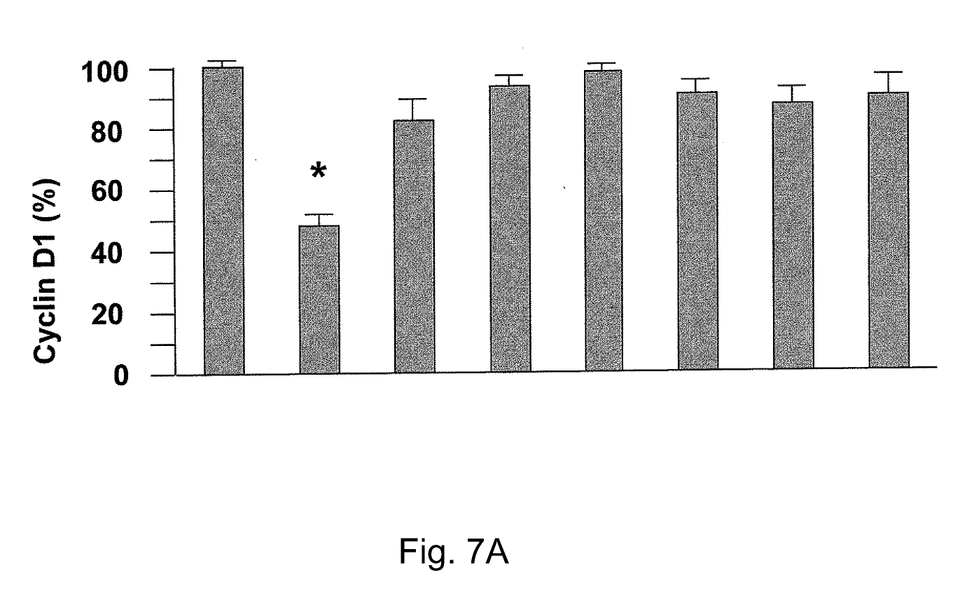

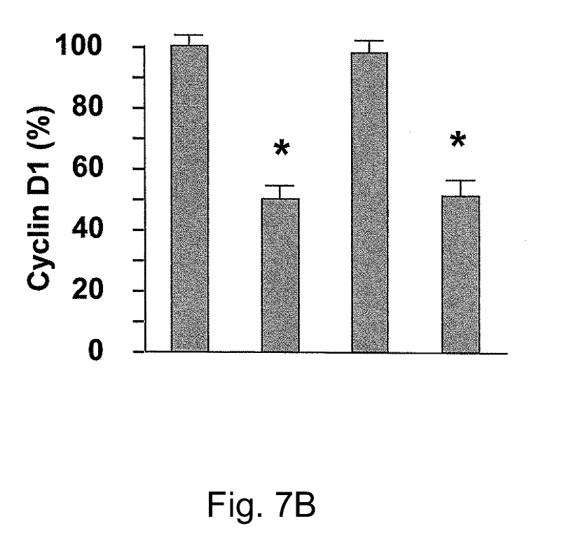

As₂O₃ induced cyclin D1 degradation in 26S and 20S proteasomes but not lysosomes. Preincubation of Jeko-1 cells with the 26S and 20S proteosome inhibitors MG132 (30 μM), bortezimab (10 μg/ml) and lactacystin (10 μM) attenuated As₂O₃-induced cyclin D1 down-regulation (FIG. 7A). However, pre-incubation with the lysosomal inhibitor ammonium chloride (NH₄Cl) had no effect on As₂O₃-induced down-regulation of cyclin D1 (FIG. 7B). The results confirmed that As₂O₃ down-regulated cyclin D1 by promoting its ubiquitination, hence targeting it to the proteosome for degradation.

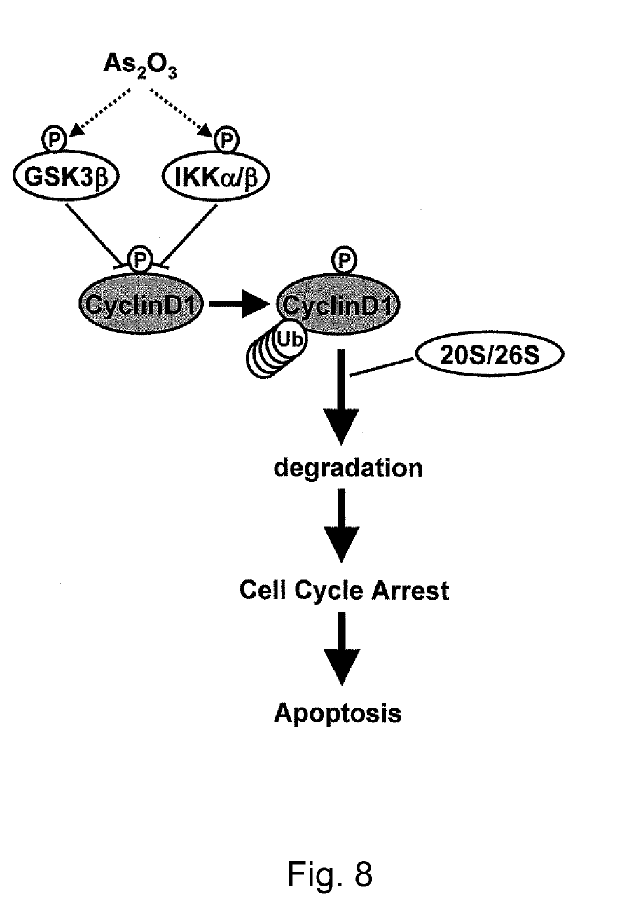

Overall model. An overall model of the action of As₂O₃ on MCL is shown in FIG. 8.

### Example 2

### Clinical Study of Oral-As₂O₃ in the Treatment of Patients with Refractory and Relapsed MCL that Over-Expressed Cyclin D1

### Materials and Methods

Patients. Consenting patients with relapsed or refractory B-cell lymphomas, and an ECOG performance status of <2 were recruited. All patients gave informed consent, and the treatment was approved by the institute review board of Queen Mary Hospital.

Treatment. Treatment was initiated with oral-As₂O₃ (10 mg/day for patients below 70 years old with normal renal function; 5 mg/day for patients over 70 years old, or with impaired renal function), ascorbic acid (AA, 1 g/day) and chlorambucil (4 mg/day) as outpatients until disease response or progression was documented. In patients with bulky disease, debulking with VPP (vincristine 2 mg/day×1, prednisolone 30 mg/day×14 and procarabzine 50-100 mg/day×14) was used. After maximum response was achieved, chlorambucil was taken off and a maintenance regimen of As₂O₃ (5-10 mg/day) and AA (1 g/day) was given for two weeks every 2 months for a planned two years. Responses were classified according to standard NCI criteria, and monitored by regular physical examination, marrow and blood assessment, and computerized tomographic scans.

### Results

Characteristics of patients with MCL. Table 1 shows the results of the clinical use of oral-As₂O₃ in patients with refractory or relapsed mantle cell lymphoma that over-expressed cyclin D1. The results showed an overall response rate of 64%. Four patients achieved complete remission (CR), whereas two patients achieved complete remission unconfirmed. Of the fourteen patients treated (Table 1), eleven had advanced relapses (R) (R2, n=5; R3, n=4; R4, n=2). Three patients treated in R1 had advanced age (76, 77 and 90 years). All but two patients had received an anthracycline based multi-agent chemotherapy. Other previous treatment included rituximab (n=8), autologous hematopoietic stem cell transplantation (HSCT) (n=3), and bortezomib (n=1). Other poor prognostic indicators included marrow infiltration (n=11) and extensive extranodal involvement (n=9), so that 12/14 (86%) cases had stage IV disease. The median time from initial diagnosis to As₂O₃ treatment was 33 (8-85) months.

TABLE 1
Clinicopathologic features and treatment outcome of 14 patients with relapsed or refractory MCL

| | | Initial disease | | | Current relapse | | | | | Outcome and |
|---|---|---|---|---|---|---|---|---|---|---|
| | | stage | sites | Previous treatment | Time* | No | Sites | Total As₂O₃ | response | survival |
| 1 | M/69 | III | Colon, abdomen | FND × 6, COPP × 6 | 56 m | 2 | Cervical | 140 mg | CR | off Rx, 28 m+. |
| 2 | M/63 | IV | BM, generalized LN | R-CEOP × 6, IMVP × 6 | 11 m | 2 | BM, cervical | 160 mg | CR | on Rx, 13 m+. |
| 3 | M/65 | IV | BM, mesentery, | FND × 7, IMVP × 2, | 85 m | 3 | Eye | 120 mg | CR | on Rx, 17 m+. |
| | | | generalized LN | R-DHAP × 8 | | | | | | |
| 4 | F/77 | IV | Pleura, generalized | Clb | 33 m | 1 | Groin, jaw | 140 mg | CR | R2 at 16 m, CR |
| | | | LN | | | | | | | again with |
| | | | | | | | | | | As₂O₃ + Clb |
| 5 | M/70 | III | Generalized LN | COPP × 2, IMVP × 6, | 85 m | 4 | Cervical, | 250 mg | CRu | R5 at 20 m, on |
| | | | | Clb | | | abdomen | | | As₂O₃ + Clb |
| 6 | M/76 | IV | BM, generalized LN | CEOP × 7 | 19 m | 1 | BM, leukemic, | 210 mg* | CRu | on Rx, 8 m+ |
| | | | | | | | eyes, | | | |
| | | | | | | | generalized LN | | | |
| 7 | M/58 | IV | BM, generalized LN | CEOP × 6, R-ESHAP × 6 | 18 m | 2 | Generalized skin | 140 mg | PR | On Rx, 3 m+ |
| 8 | M/81 | IV | BM, leukemic, liver, | CHOP × 6, ChlVPP × 2 | 18 m | 2 | BM, LN, liver, | 300 mg* | PR | died at 16 m |
| | | | spleen | | | | spleen, leukemic | | | |
| 9 | M/51 | IV | Generalized LN, spleen, | CVAD × 7, CEOP × 2, | 25 m | 4 | BM, LN, scalp | NA* | Static | on Rx, 8 m+ |
| | | | BM, scalp, eye | R-DHAP × 3, Thal | | | | | | |
| 10 | F/76 | IV | General LN, BM, scalp | R-COPP × 6 | 12 m | 2 | BM, LN | 160 mg* | PR | died at 6 m |
| 11 | M/90 | IV | BM, leukemic | Clb | 8 m | 1 | BM, leukemic | NA | NR | died at 4 m |
| 12 | M/54 | IV | Generalized LN, BM, | CEOP × 6, DHAP × 1, | 36 m | 3 | BM, generalized | NA | Static | died at 17 m |
| | | | gut, liver, | NOPP × 5, Clb | | | LN, spleen | | | |
| | | | spleen, leukemic | | | | | | | |
| 13 | F/57 | IV | Generalized LN, BM, | CEOP × 6, DHAP × 4, | 36 m | 3 | BM, LN | NA | NR | died at 1 m |
| | | | spleen | R-BVP × 3 | | | | | | |
| 14 | M/63 | IV | Generalized LN, | CEOP × 6, AHSCT, | 72 m | 3 | BM, generalized | NA | NR | died at 1 m |
| | | | pleura, BM | R-DHAP × 6, | | | LN | | | |
| | | | | Thal, velcade, FND | | | | | | |

M: male; F: female; LN: lymphadenopathy; BM: bone marrow; m: months; R: rituximab; CEOP: cyclophospamide, epirubicin, vincristine, predniolone
FND: fludarabine, mitoxantrone, dexamethasone; DHAP: cisplatinum, cytosine arabinoside, dexamethasone; Thal: thalidomide
ChlvPP: chlorambucil, vincristine, procarbazine, prednisolone; COPP: cyclophosphamide, vincristine, procarbazine, prednisolone;
NOPP: mitoxantrone; vincristine, procarbazine, prednisolone; BVP; bleomycin, vinblastine, prednisolone; AHSCT: autologous hematopoietic stem cell transplantation
Clb: chlorambucil; NA: not available; CR: complete remission; CRu: complete remission (unconfirmed); PR: partial remission; NR; no response

Treatment response. Nine patients responded, giving an OR rate of 64%. Four patients (cases 1-4) achieved CR. Two patients (cases 5, 6) achieved unconfirmed CR (CRu). They had become asymptomatic without any detectable superficial diseases. Marrow and peripheral blood involvement was also cleared. However, small residual internal lymph nodes remained, These lymph nodes were negative on gallium scan and had remained static in size. Three patients had partial responses (PR) with >50% reduction in the size of assessable lymph nodes.

Case 6 had bilateral orbital infiltration at relapse that completely resolved after 4 months of oral As₂O₃ treatment and ascorbic acid. Case 8 who was relapsing in leukemic phase with massive splengomegaly showed partial remission after 8 months of treatment with oral As₂O₃ and ascorbic acid as determined by MRI scans. Histological analysis revealed that case 8 had dense marrow infiltration that resolved after 8 months of treatment with oral-As₂O₃ and ascorbic acid.

Outcome. Of the four patients with CR, one had relapsed at 16 months. She achieved a CR3 again with daily As₂O₃ and resumption of chlorambucil. Two patients were still on maintenance As₂O₃+AA treatment, while one had completed the planned two years of treatment. Of the two patients with CRu, one patient had relapsed at 20 months. He achieved CR5 again with As₂O₃ and chlorambucil therapy. For the three patients with PR, one patient developed progressive disease while on maintenance therapy 12 months later and died of refractory lymphoma. Two defaulted treatment and both relapsed, Toxicity. Significant (W.H.O grade 3-4) neutropenia and thrombocytopenia was observed in 7 patients. These patients had previously received multiple chemotherapy, or autologous HSCT. The neutropenia responded to hematopoietic growth factors. No significant sepsis or bleeding were observed. Other side effects included fever (n=7), herpes zoster reactivation (n=3), fluid accumulation (n=2), nausea (n=3) and headache (n=2). No significant QT prolongation or arrhythmia was observed. Five patients did not report any side effects at all.

As₂O₃ suppresses MCL cell growth by targeting cyclin D1. As₂O₃ induces the phosphorylation of GSK-3β and IKK. Cyclin D1 over-expression is pathogenetically important in a vast diversity of cancers. Oral-As₂O₃ inhibited refractory or relapsed MCL in 14 patients, which over-expressed cyclin D1, with an overall response in 9 patients (64%). Four patients achieved complete remission, two patients complete remission unconfirmed, and three patients with partial remissions. These results were very good, given that these patients had refractory or relapsed disease.

Taken together, the evidence demonstrates that As₂O₃ decreases cyclin D1 and that the decrease in cyclin D1 was post-transcriptional. As₂O₃ induces GSK-3β and IKK activation and hence phosphorylation of cyclin D1. Phosphorylated cyclin D1 is degraded in the proteasome. Oral As₂O₃ induces a high response rate clinically in patients with refractory or relapsed MCL, a cancer that over-expresses cyclin D1.

We claim:

1. A method for inhibiting cyclin D1 production in a cell, comprising contacting the cell with an amount of arsenic trioxide effective to inhibit cyclin D1 production therein.

2. The method according to claim 1, wherein the cell is a cancer cell.

3. The method according to claim 2, wherein the cell is from a cancer of the digestive tract, cancer of the female genital tract, and malignant lymphomas.

4. The method according to claim 2, wherein the cancer cells are in a patient and the arsenic trioxide is administered orally.

5. The method according to claim 4, wherein the cancer cell is from a human female genital tract cancer, a digestive tract cancer, or a malignant lymphoma.

6. The method according to claim 4 wherein the cell is a human mantle cell lymphoma (MCL).

7. A unit dosage form for oral administration comprising arsenic trioxide in a pharmaceutically acceptable carrier for enteral administration.

8. The unit dosage form of claim 7, farther comprising one or more pharmaceutically acceptable excipients.

9. The unit dosage form of claim 7, wherein the arsenic trioxide is present in an amount from 5 to 10 mg.

10. The unit dosage form of claim 7, wherein the unit dosage form is selected from the group consisting of solutions, suspensions, emulsions, syrups, tablets, and capsules.

11. A unit dosage form for oral administration comprising arsenic trioxide in a pharmaceutically acceptable carrier for enteral administration, wherein the dosage form contains a sufficient amount of arsenic trioxide to deliver a dose in the range of 5 to 10 mg.

12. The unit dosage form of claim 11, further comprising one or more pharmaceutically acceptable excipients.

13. The unit dosage form of claim 11, wherein the unit dosage form is selected from the group consisting of solutions, suspensions, emulsions, syrups, tablets, and capsules.
